(19) United States
(12) Patent Application Publication
(10) Pub. No.: US 2004/0082071 A1
(43) Pub. Date: Apr. 29, 2004

(54) Apparatus for high-throughput analysis of membranes

(76) Inventor: Ilya Feygin, Mountainside, NJ (US)

Correspondence Address: DEMONT & BREYER, LLC, SUITE 250, 100 COMMONS WAY, HOLMDEL, NJ, 07733, US

(21) Appl. No.: 10/638,829

(22) Filed: Aug. 11, 2003

Related U.S. Application Data
(63) Continuation-in-part of application No. 10/139,766, filed on May 6, 2002.

(51) Int. Cl. G01N 33/00

(52) U.S. Cl. 436/8; 422/68.1

(57) ABSTRACT

An apparatus for high-throughput analysis of membranes. In some embodiments, the apparatus comprises one or more cartridges that are disposed on a carrier. Within at least some of the cartridges are at least two, selectively-permeable barriers. The barriers are impermeable to fluid except when a pressure differential exists across them. A chamber is defined between the barriers. A membrane, such as a sample of live tissue, is disposed within the chamber. In some embodiments, the membrane bifurcates the chamber into two, isolated compartments. There is an opening at each of the ends of the cartridge. At least two feed fittings, which detachably engage those openings, are coupled to a feed system, a sampling and analysis system, or both. Either the carrier or feed fittings are moved to sequentially move the feed fittings and a cartridge into alignment.

### STATEMENT OF RELATED CASES

This application is a continuation-in-part of co-pending U.S. patent application Ser. No. 10/139,766 filed May 6, 2002.

### FIELD OF THE INVENTION

The present invention relates generally to an apparatus for high-throughput analysis of membranes, such as animal tissues.

### BACKGROUND OF THE INVENTION

Apparatuses for conducting “ADMETox” (i.e., absorption, distribution, metabolism, excretion, and toxicology) and other studies on membranes (e.g., live tissue, etc.) have been disclosed in U.S. pat. appls. Ser. Nos. 10/139,766 and 10/225,057, both of which are incorporated by reference herein. In some embodiments, those apparatuses comprise a plurality of self-sealing, multi-chamber enclosures or cartridges and a fluidic control system. The fluidic control system, which couples to the cartridges, includes a “supply side,” which is a feed system for delivering fluids (e.g., reagents, buffers, etc.) to the cartridges, and a “receiving side,” which is a sampling and analysis system for analyzing fluid samples that are withdrawn from the cartridges.

In an illustrative embodiment, each cartridge has a two-piece housing with openings disposed at remote ends thereof. Each opening receives a feed fitting. The feed fittings function to seal the cartridge and provide an interface to the external environment. With regard to the interface function, in some embodiments, each feed fitting receives a plurality of conduits, some of which couple to the feed system, and others of which couple to the analysis system.

In some embodiments, these apparatuses are fully automated (e.g., as to the introduction of fluids, withdrawal of samples, the analysis of the samples, etc.). The apparatuses are capable of operating at high speeds that are substantially faster than most devices for conducting ADMETox testing.

While the apparatuses described in U.S. pat. appls. Ser. Nos. 10/139,766 and 10/225,057 are an improvement over earlier ADMETox systems, they do require a large number of valves, pumps, etc. In fact, the number of valves required is directly proportional to the number of cartridges, the number of compounds being tested, and the number of analytical interfaces.

### SUMMARY

The illustrative embodiment of the present invention is an apparatus for high-throughput analysis of membranes that avoids some of the problems of the prior art. In some embodiments, the apparatus includes far less valve and pumps than are required in prior art apparatuses, such as those described in U.S. pat. appls. Ser. Nos. 10/139,766 and 10/225,057.

In accordance with the illustrative embodiment, the apparatus comprises one or more cartridges that are disposed on a carrier. Within at least some of the cartridges are at least two, selectively-permeable barriers, one of which is disposed proximal to a first end of the cartridge and the other of which is disposed proximal to a second end of the cartridge. The region between the barriers defines a chamber. The barriers are impermeable to fluid except when a pressure differential exists across them. There is an opening at each of the first end and second end of the cartridge.

A membrane, such as a sample of live tissue, is disposed within the chamber, in the cartridge. In some embodiments, the membrane bifurcates the chamber into two, isolated compartments.

In the illustrative embodiment, the apparatus also includes two feed fittings. One of the feed fittings is disposed on one side of the carrier and the other feed fitting is disposed on the other side of the carrier. The feed fittings receive conduits that are coupled to a feed system, a sampling and analysis system, or both.

In some embodiments, the carrier moves, and as it does, cartridges are sequentially registered with the two feed fittings. In some other embodiments, the feed fittings are moved, and they are sequentially registered with the cartridges. When registered, one of the feed fittings is positioned to be received by the opening at the first end of the cartridge and the other feed fitting is positioned to be received by the opening at the second end of the cartridge.

Actuator(s) move the feed fittings into engagement with the cartridge. Once the feed fittings are engaged to the cartridge, fluid can be delivered to the chamber via one or both feed fittings and fluid can be removed from the chamber via one or both of the feed fittings. But the conduits from the feed fittings do not penetrate the selectively-permeable barriers. Rather, adding or removing fluid from the chamber is effected by creating a pressure differential across one or both of the selectively-permeable barriers.

In particular, as fluid is delivered to cartridge, pressure builds against the selectively-permeable barrier. As the pressure differential across the barrier increases, fluid passes through the barrier into the chamber. Likewise, to withdraw fluid from the chamber, pressure is reduced against the selectively-permeable barrier, such as by generating a vacuum-inducing flow in one of the conduits. As the pressure differential increases, with higher pressure within the chamber, fluid crosses the selectively-permeable barrier and can be sampled by the conduits.

### BRIEF DESCRIPTION OF THE DRAWINGS

FIG. 1 depicts an apparatus for high-throughput analysis of membranes in accordance with the illustrative embodiment of the present invention.

FIG. 2A depicts an exploded, cross-sectional view of a cartridge and feed fittings for use in conjunction with the illustrative embodiment of the present invention.

FIG. 2B depicts the cartridge of FIG. 2A in an assembled state, and containing a membrane.

FIG. 3A depicts the cartridge of FIG. 2B within a carrier, as in FIG. 1.

FIG. 3B depicts feed fittings engaged to the cartridge of FIG. 3A.

FIG. 4 depicts a rubber bushing for retaining the cartridge of FIG. 2B in the carrier of FIG. 1.

FIG. 5 depicts a variation of a carrier for use in conjunction with the illustrative embodiment of the present invention.

FIG. 6 depicts a variation of the manner in which a membrane is enclosed by the cartridge of FIG. 2B.

FIG. 7 depicts a variation of the carrier shown in FIG. 1.

### DETAILED DESCRIPTION

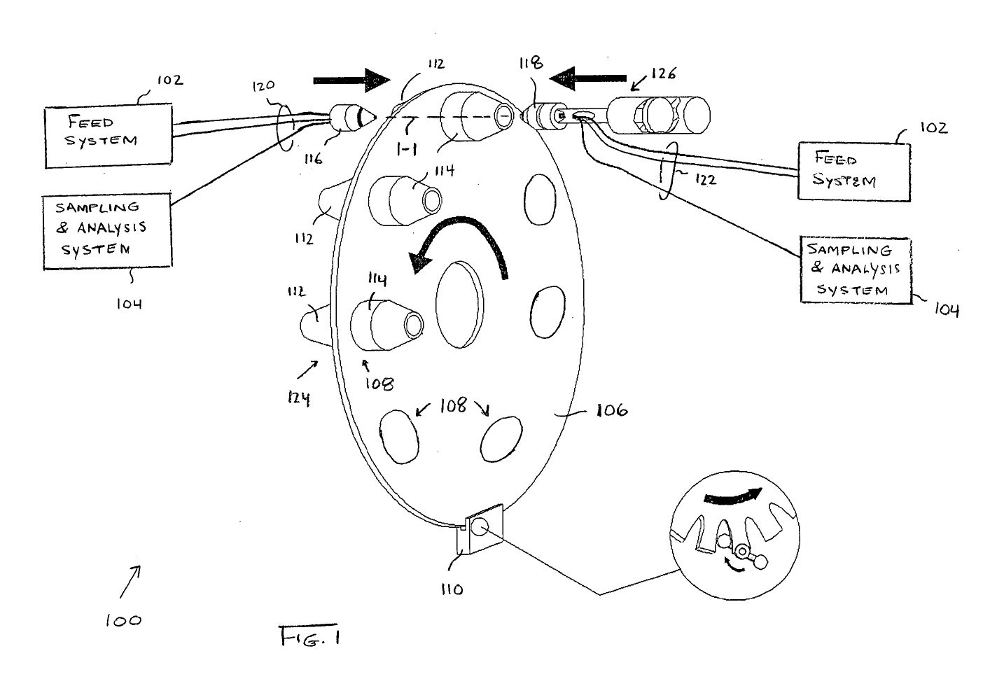

FIG. 1 depicts apparatus 100 for high-throughput analysis of membranes, tissues, etc., in accordance with the illustrative embodiment of the present invention. Apparatus 100 includes feed system 102, sampling and analysis system 104, carrier 106, carrier drive 110, first cartridge housing 112, second cartridge housing 114, first feed fitting 116, second feed fitting 118, first plurality of conduits 120, and second plurality of conduits 122, inter-related as shown. Note that the term “cartridge,” as used herein, refers to the same element as the “multi-chamber enclosure” of U.S. pat. apps. Ser. Nos. 10/139,766 and 10/225,057.

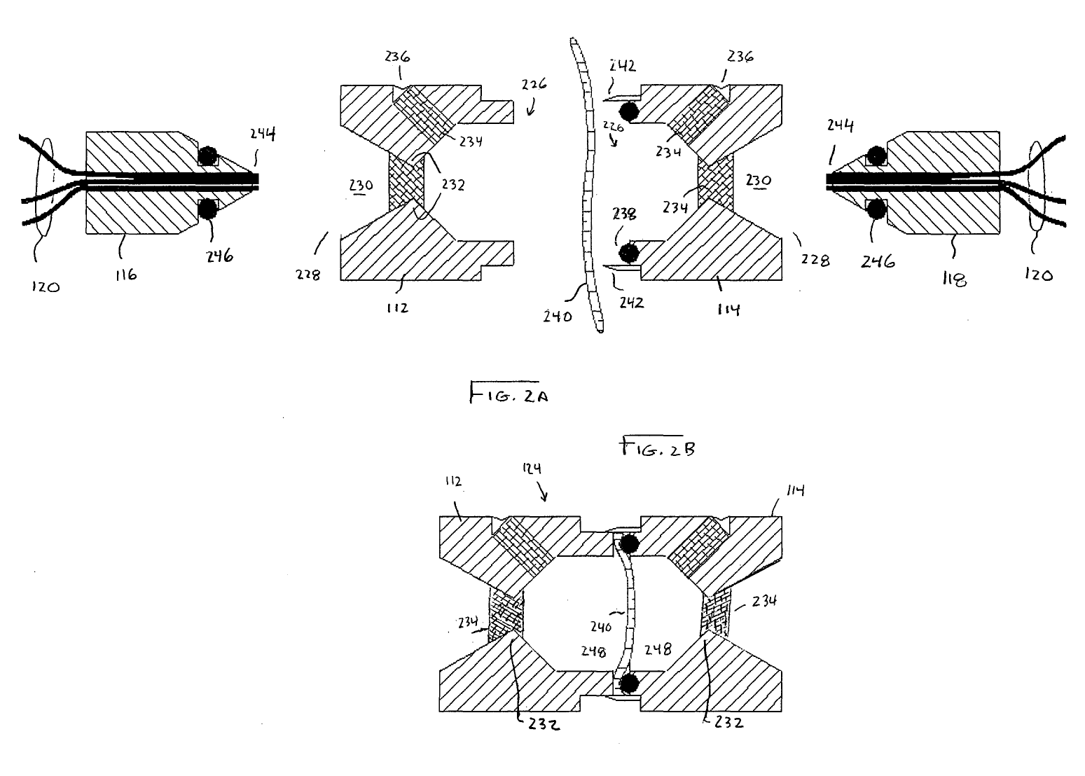

As depicted in FIG. 1, a plurality of cartridges 124 are arrayed on carrier 106. The cartridges are positioned in a plurality of openings or holes 108 that are disposed along a marginal region of carrier 106. In the illustrative embodiment, each cartridge comprises two housings: first cartridge housing 112 and second cartridge housing 114. The housings protrude beyond the major surfaces of carrier 106. A membrane (see, e.g., FIGS. 2A and 2B) is disposed within at least some of cartridges 124.

FIG. 2A provides further detail of cartridge 124 and feed fittings 116 and 118. In FIG. 2A, cartridge 124 is depicted before assembly (i.e., before housings 112 and 114 are engaged to one another).

As depicted in FIG. 2A, each housing 112 and 114 has an opening at both ends. In the illustrative embodiment, those openings are relatively larger opening 226 and relatively smaller opening 228. In some other embodiments, the position of the relatively larger and smaller openings is reversed, and in yet some other embodiments, the openings are the same size as one another.

In the illustrative embodiment, relatively smaller opening 228 leads to feed-fitting receiving region 230, which has a conical shape. Selectively-permeable material 234 is disposed at constriction 232. The operation of selectively-permeable material 234 is described below. Beyond constriction 232, the interior of the housings widen again. Each housing also includes vent 236. In the illustrative embodiment, selectively-permeable material 234 is disposed in the vent. O-ring 238 provides a leak-proof seal.

Also depicted in FIG. 2A is membrane 240. In some embodiments, membrane 240 is live tissue. In oral absorption studies, membrane 240 is typically intestinal tissue, or, alternatively, the mucosa of the intestinal tissue, stripped from underlying layers. In other absorption studies, skin, buccal, nasal, pulmonary, corneal, vaginal or other tissues can suitably be used. Or, as desired, membrane 240 can be a tissue substitute (i.e., synthetic tissue), such as nitrocellulose, nylon, polypropylene, etc. Furthermore, membrane 240 can be a mono-layer of cells from a cell line that is grown on the surface of a tissue or a tissue substitute.

Blades 242, which in the illustrative embodiment are depicted on housing 114, size membrane 240 as housings 112 and 114 are brought together.

FIG. 2A also shows feed fittings 116 and 118. The feed fittings have a conically-shaped portion that fits into feed-fitting receiving region 230. Conduits 120 pass through feed fitting 116 and terminate at or near apex 244. Feed fittings include o-ring 246 to create a leak-proof seal between feed-fitting receiving region 230 and feed fittings 116 and 118.

FIG. 2B depicts housings 112 and 114 engaged to one another sandwiching sized membrane 240. Chamber 248 is defined within cartridge 124 between selectively permeable material 234 that is located in constriction 236 within each housing. Membrane 124 bifurcates chamber 248. This arrangement can be used to study membrane transport, among other parameters.

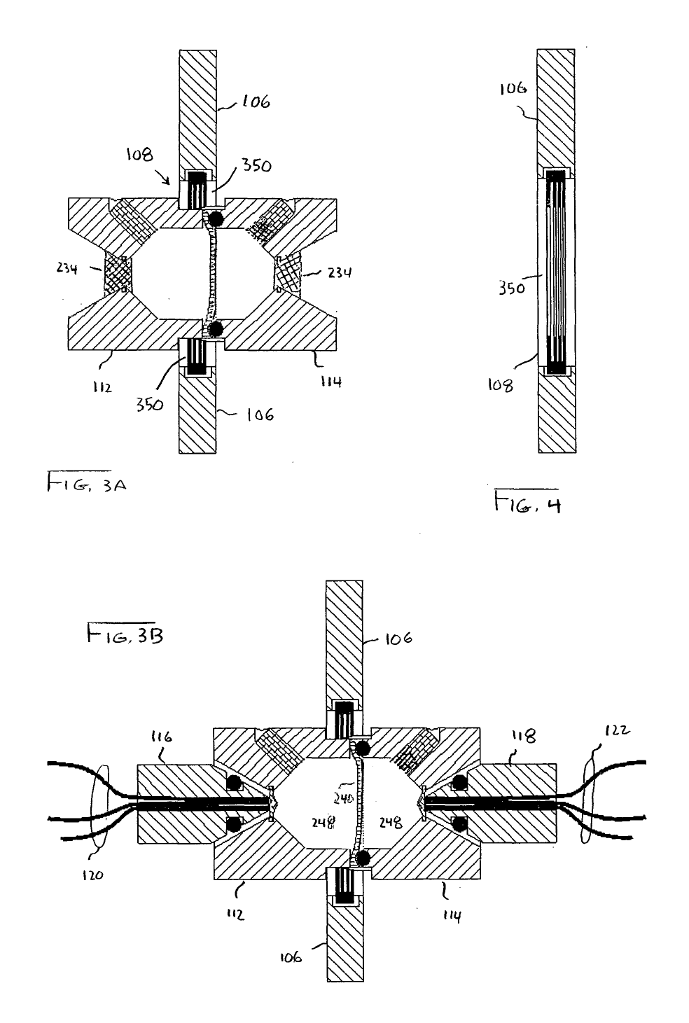

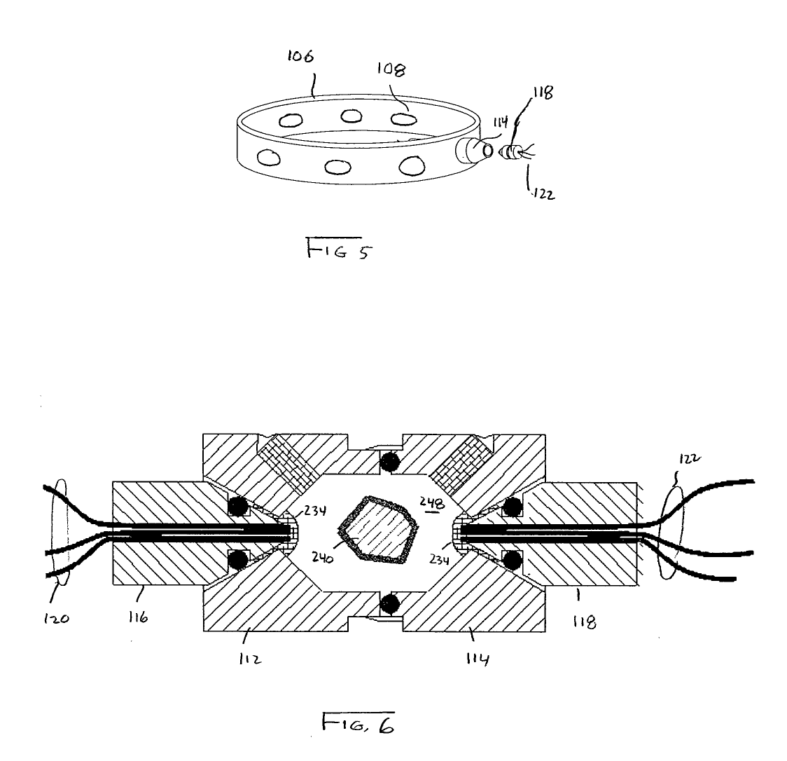

After cartridge 124 is assembled, it is inserted into a hole 108 in carrier 106. At this point, as explained further below, feed fittings 116 and 118 are not engaged to cartridge 124. FIG. 3A depicts a cross-sectional view of a portion of carrier 106, with cartridge 124 inserted through one of holes 108, as in FIG. 1. To retain cartridge 124 in hole 108, a rubber bushing 350 is used. Any of a variety of other arrangements for retaining cartridges 124 in holes 108 may suitably be used. FIG. 4 depicts bushing 350 inserted within hole 108 (sans cartridge 124).

Referring again to FIG. 1, first feed fitting 116 is disposed on one side of carrier 106 and second feed fitting 118 is disposed on the other side of the carrier. In the illustrative embodiment, feed fittings 116 and 118 are positioned to align with respective cartridge housings 112 and 114 along axis 1-1. Carrier drive 110 turns carrier 106 so that cartridges 124 are sequentially moved to into position along axis 1-1. In the illustrative embodiment, carrier drive 110 is implemented as a “Geneva mechanism,” but any one of a variety of arrangements known to those skilled in the art for driving carrier 106 may suitably be used.

Once a cartridge 124 is at location 128, first feed fitting 116 is moved into engagement with housing 112 and second feed fitting 118 is moved into engagement with housing 114. Feed fittings 116 and 118 are received within feed-fitting receiving region 230 of each housing. The feed fittings are moved into position by actuators, such as piston actuator 126 (only shown for feed fitting 118).

FIG. 3B depicts cartridge 124 in carrier 106 with feed fittings 116 and 118 inserted into feed-fitting receiving region 230 of each housing 112 and 114 of cartridge 124.

With continuing reference to FIGS. 1 and 3B, once feed fittings 116 and 118 are engaged to cartridge 124, fluids can be delivered to either side of chamber 248 or withdrawn from either side thereof. More particularly, conduits 120, which are coupled to first feed fitting 116, are coupled to feed system 102 and sampling and analysis system 104. Similarly, conduits 122, which are coupled to first feed fitting 118, are likewise coupled to feed system 102 and sampling and analysis system 104.

It can be seen that the conduits 120 and 122 are prevented from direct contact with chamber 248 by selectively permeable material 234. When there is no pressure differential across the selectively permeable material, it is impermeable to fluids. In other words, there is no transport between the external environment and chamber 248. When, however, a pressure differential exists, fluids will pass through selectively permeable material 234 in the direction of lower pressure.

When, for example, fluid is delivered from feed system 102 through one or more of conduits 120 and first feed fitting 116 to selectively permeable material 234, the pressure within feed-fitting receiving region 230 rises above the pressure within chamber 248. This causes the fluid to pass through selectively permeable material 234 into chamber 248. The same is true for fluid that is delivered from feed system 102 through one or more conduits 122 and second feed fitting 118.

To sample from either side of chamber 248, a vacuum is drawn on at least one of conduits 120 (or 122). This creates a pressure differential across selectively-permeable material 234, so that fluids are passed from chamber 248 to the vacuum creating conduit 120 (or 122).

In some embodiments, selectively permeable membrane 234 is a material, such as silicon, that is cut at an angle to the normal (e.g., like gill slits). If a pressure differential exists across the disc, the slits will “open,” due to the higher pressure on one side. When the slits open, the disc will pass fluid. In the absence of a pressure differential, the cuts will remain closed. A disc having a thickness of about ⅛ inch and appropriately dimensioned to fit within the interior of housings 112 and 114 will be suitable for this purpose.

In some other embodiments, selectively permeable membrane 234 is a filtering material, such as is commercially available from Whatman, PALL and others. For example, the Whatman “polydisc” PM2.5 filters are suitable for use as selectively permeable membrane 234. Technical specifications for these filters are provided below. See, http://www.whatman.com.

Technical Specifications

| | | | | | | Flow Rate (1) | |
|---|---|---|---|---|---|---|---|
| | | | | | | Methanol | Flow Rate (1) |
| | | Integrity Test Data (1) | | Integrity Test Data (1) | | mL/min | Air SLPM |
| | Pore | Methanol Bubble Point | | Water Breakthrough | | at 0.7 bar | at 0.2 bar |
| Product | Size | bar | psi | bar | psi | (10 psi) | (3 psi) |
| Polydisc | 0.1 μm | 1.7 | 25 | 3.4 | 50 | 200 | 8 |
| TF | | | | | | | |
| Polydisc | 0.2 μm | 0.9 | 13 | 2.1 | 30 | 400 | 16 |
| TF | | | | | | | |
| Polydisc | 0.45 μm | 0.5 | 7 | 1.1 | 16 | 700 | 24 |
| TF | | | | | | | |
| Polydisc | 1.0 μm | 0.2 | 3 | 0.3 | 5 | 900 | 30 |
| TF | | | | | | | |

In yet some further embodiments, specially treated open-cell foams can be used. These foams retain liquid until differential pressure is applied.

To the extent that different fluids are being delivered to various cartridges 124 on carrier 106, conduits 120 and 122 must be cleaned between dispensations. This can be accomplished in a variety of ways. For example, membrane-containing cartridges 124 can be alternated with a “cleansing” cartridge. The fluids (from feed system 102) that are intended for the next membrane-containing cartridge 124 can be delivered first to a cleansing cartridge. Thus, when fluid is ready to be delivered to membrane-containing cartridge 124, conduits 120 and 122 have already been purged with those fluids. Likewise, a cleaning solution can be contained in a cleansing cartridge such that the fluid can be withdrawn through conduits 120 and 122. A variety of known schemes for cleansing can suitably be used.

It is to be understood that the above-described embodiments are merely illustrative of the present invention and that many variations of the above-described embodiments can be devised by those skilled in the art without departing from the scope of the invention.

For example, while in the illustrative embodiment, carrier 106 is depicted as a flat disc, in some other embodiments, the carrier has a different shape or structure. For example, in a variation that is depicted in FIG. 5, carrier 106 is implemented as a ring, which is positioned horizontally. In still further variations, carrier 106 has a polygonal shape of n sides, wherein n=4 to 8.

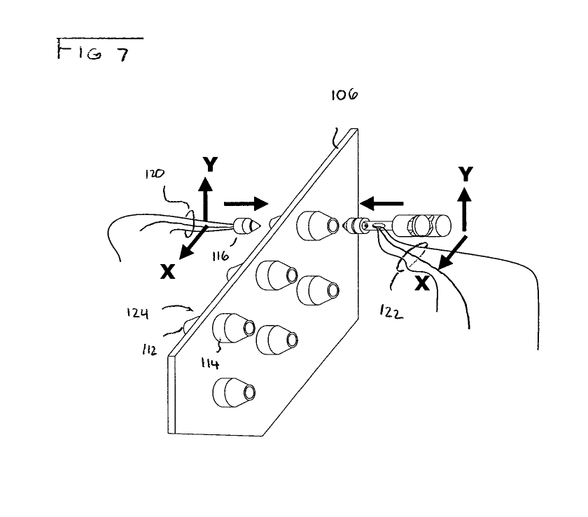

And in another variation of the illustrative embodiment, rather than bifurcating chamber 248 into sections, a membrane is disposed loosely within the chamber, as depicted in FIG. 6. This arrangement enables flow-through testing, such as for toxicology studies.

In some variations of the illustrative embodiment, holes 108 and cartridges 124 are disposed inward of the marginal region shown in FIG. 1. In some other variations, cartridges are disposed in radial arrays on carrier 106. In yet some further variations, the feed fittings, rather than carrier, are moved into registration with the cartridges. Of course, a drive system for the feed fittings is required for such variations. Such drive systems are well known to those skilled in the art.

In yet some additional variations, carrier 106 is a rectangular plate with cartridges 124 disposed in a rectangular array on it. An illustrative rectangular carrier is depicted in FIG. 7. Either carrier 106 or feed fittings 116 and 118 are moved in the x and y directions so that different cartridges are registered to feed fittings 116 and 118. In some embodiments, the cartridges are disposed in a rectangular 8×12 array.

In still some further embodiments, more than two feed fittings are used to speed-up testing. For example, in some embodiments, a linear array of feed fittings is used, wherein the array has m pairs of feed fittings for coupling to cartridges 124. The carrier or feed fittings are then moved linearly into registration with successive rows (or columns) of cartridges, each having m cartridges.

It is therefore intended that such variations, and others that will occur to those skilled in the art in view of the present disclosure, be included within the scope of the following claims and their equivalents.

I claim:

1. An apparatus comprising a cartridge, wherein said cartridge comprises:
a first end and a second end;
a first opening at said first end and a second opening at said second end; and
a first selectively-permeable material disposed proximal to said first opening and a second selectively-permeable material disposed proximal to said second opening.

2. The apparatus of claim 1 further comprising:
a carrier, wherein:
said carrier has a first major surface and a second major surface;
said carrier has a plurality holes that extend through said first major surface and said second major surface;
said cartridge is disposed within one of said holes;
said first opening is accessible proximal to said first major surface; and
said second opening is accessible proximal to said second major surface.

3. The apparatus of claim 2 further comprising a carrier drive, wherein said carrier drive causes said carrier to move.

4. The apparatus of claim 2 further comprising a first feed fitting and a second feed fitting, wherein:
said first feed fitting is proximal to said first major surface and is sized and configured to be received by said first opening at said first end of said cartridge;
said second feed fitting is proximal to said second major surface and is sized and configured to be received by said second at said second end of said cartridge; and
movement of at least one of either said carrier or said first and second feed fittings aligns:
said first opening of said cartridge and said first feed fitting; and
said second opening of said cartridge with said second feed fitting.

5. The apparatus of claim 4 wherein a chamber is defined within said cartridge between said first selectively-permeable membrane and said second selectively permeable membrane.

6. The apparatus of claim 5 further comprising a first plurality of conduits, wherein said first plurality of conduits pass through said first feed fitting into said first end of said cartridge, but not into said chamber, and wherein at least one of said conduits is coupled to a feed system for conducting fluid to said first end of said cartridge.

7. The apparatus of claim 5 further comprising a first plurality of conduits, wherein said first plurality of conduits pass through said first feed fitting into said first end of said cartridge, but not into said chamber, and wherein at least one of said conduits is coupled to a sampling and analysis system for withdrawing fluid from said chamber proximal to said first end of said cartridge.

8. The apparatus of claim 6 wherein at least one of said conduits is coupled to a sampling and analysis system for withdrawing fluid from said chamber proximal to said first end of said cartridge.

9. The apparatus of claim 8 wherein said sampling and analysis system comprises analytical equipment for analyzing said fluid that was withdrawn from said chamber.

10. The apparatus of claim 4 further comprising a second plurality of conduits, wherein said second plurality of conduits pass through said second feed fitting into said second end of said cartridge, but not into said chamber, and wherein at least one of said conduits is coupled to a feed system for conducting fluid to said second end of said cartridge.

11. The apparatus of claim 4 further comprising a second plurality of conduits, wherein said second plurality of conduits pass through said second feed fitting into said second end of said cartridge, but not into said chamber, and wherein at least one of said conduits is coupled to a sampling and analysis system for withdrawing fluid from said chamber proximal to said second end of said cartridge.

12. The apparatus of claim 11 wherein said sampling and analysis system comprises analytical equipment for analyzing said fluid that was withdrawn from said chamber.

13. The apparatus of claim 10 wherein at least one of said conduits is coupled to a sampling and analysis system for withdrawing fluid from said chamber proximal to said second end of said cartridge.

14. The apparatus of claim 13 wherein said sampling and analysis system comprises analytical equipment for analyzing said fluid that was withdrawn from said chamber.

15. The apparatus of claim 4 further comprising a membrane, wherein said membrane is disposed within said chamber.

16. The apparatus of claim 15 wherein said membrane bifurcates said chamber.

17. An apparatus comprising:
a carrier;
a plurality of cartridges, wherein:
said plurality of cartridges are coupled to said carrier;
each cartridge comprises two selectively-permeable barriers;
said selectively-permeable barriers are spaced from one another defining a chamber therebetween;
a first feed fitting and a second feed fitting, wherein:
said first feed fitting is coupled to at least one of a feed system and a sampling and analysis system;
said second feed fitting is coupled to at least one of a sampling and an analysis system and a feed system; and
said first feed fitting and said second feed fitting are physically adapted to removably couple to said cartridges.

18. The apparatus of claim 17 wherein said first feed fitting and said second feed fitting align with an axis, and further comprising a drive system for moving said carrier, wherein, as said carrier moves, cartridges are sequentially registered with said axis for engagement with said first feed fitting and said second feed fitting.

19. The apparatus of claim 17 further comprising a drive system for moving said first feed fitting and said second feed fitting into alignment with said plurality of cartridges for engagement therewith.

20. The apparatus of claim 17 further comprising a membrane, wherein said membrane is disposed in said chamber.
